(19) United States
(12) Patent Application Publication
(10) Pub. No.: US 2018/0036292 A1
(43) Pub. Date: Feb. 8, 2018

(54) PHARMACEUTICAL COMPOSITIONS FOR THE TREATMENT OF BACTERIAL INFECTIONS

(71) Applicant: WOCKHARDT LIMITED, Chikaithana, Aurangabad (IN)

(72) Inventors: Mahesh Vithalbhai PATEL, Aurangabad 3, (IN); Sachin Subhash BHAGWAT, Aurangabad 5 (IN); Rajesh CHAVAN, Aurangabad - 6 (IN); Anusuya PATEL, Aurangabad (IN)

(73) Assignee: WOCKHARDT LIMITED, Chikaithana, Aurangabad (IN)

(21) Appl. No.: 15/550,772

(22) PCT Filed: Jan. 12, 2017
(86) PCT No.: PCT/IB2017/050154

§ 371 Date: Aug. 12, 2017

(30) Foreign Application Priority Data
Jan. 12, 2016 (IN) 201621001035

(51) Int. Cl. A61K 31/433; A61K 31/365; A61K 31/505; A61K 31/7048

(52) U.S. Cl. CPC A61K 31/433; A61K 31/7048; A61K 31/365; A61K 31/505

(57) ABSTRACT

The methods of treating bacterial infection by administering a compound of Formula (I) or a stereoisomer or a pharmaceutically acceptable derivative thereof are disclosed.

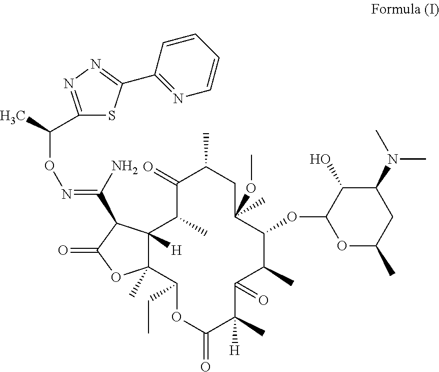 [SMILES: CC[C@@H]1OC(=O)[C@H](C)C(=O)[C@H](C)[C@@H](OC2O[C@H](C)C[C@H](N(C)C)[C@H]2O)[C@](C)(OC)C[C@@H](C)C(=O)[C@H](C)[C@H]2[C@H](/C(N)=N/O[C@@H](C)c3nnc(-c4ccccn4)s3)C(=O)O[C@@]21C]

### RELATED PATENT APPLICATIONS

This application claims benefit of Indian Patent Application No. 201621001035 filed on Jan. 12, 2016, the disclosures of which are incorporated herein by reference in its entirety as if fully rewritten herein.

### FIELD OF THE INVENTION

The invention relates to a method of treating bacterial infection in a subject.

### BACKGROUND OF THE INVENTION

Bacterial infections continue to remain one of the major causes of human diseases. A variety of antibacterial compounds are currently used in treating infections caused by bacteria. PCT International Patent Application Number PCT/IB2011/050464 discloses several compounds having antibacterial activity, including the compound of Formula (I).

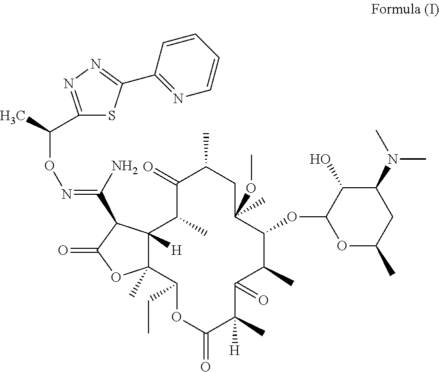 [SMILES: CC[C@@H]1OC(=O)[C@H](C)C(=O)[C@H](C)[C@@H](OC2O[C@H](C)C[C@H](N(C)C)[C@H]2O)[C@](C)(OC)C[C@@H](C)C(=O)[C@H](C)[C@H]2[C@H](/C(N)=N/O[C@@H](C)c3nnc(-c4ccccn4)s3)C(=O)O[C@@]21C]

The present invention describes a method of treating bacterial infection in a subject, said method comprising administering to said subject a compound of Formula (I) or a stereoisomer or a pharmaceutically acceptable derivative thereof.

### SUMMARY OF THE INVENTION

Accordingly, there is provided a method of treating bacterial infection in a subject, said method comprising administering to said subject a compound of Formula (I) or a stereoisomer or a pharmaceutically acceptable derivative thereof.

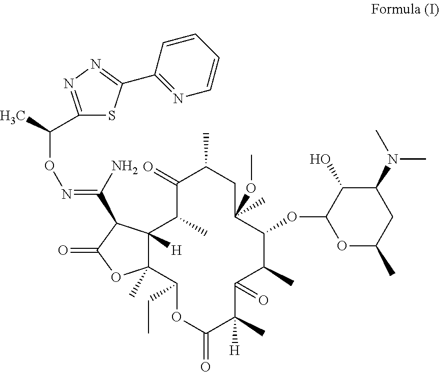 [SMILES: CC[C@@H]1OC(=O)[C@H](C)C(=O)[C@H](C)[C@@H](OC2O[C@H](C)C[C@H](N(C)C)[C@H]2O)[C@](C)(OC)C[C@@H](C)C(=O)[C@H](C)[C@H]2[C@H](/C(N)=N/O[C@@H](C)c3nnc(-c4ccccn4)s3)C(=O)O[C@@]21C]

In one general aspect, there is provided a method of treating a bacterial infection in a subject, said method comprising administering to said subject a compound of Formula (I) or a stereoisomer or a pharmaceutically acceptable derivative thereof in an amount between about 200 mg to about 2000 mg per day, for about 1 to about 10 days.

In yet another general aspect, there is provided a method of treating a bacterial infection in a subject, said method comprising oral administration to said subject a compound of Formula (I) or a stereoisomer or a pharmaceutically acceptable derivative thereof in an amount between about 400 mg to about 1200 mg per day, for about 1 to about 7 days.

The details of one or more embodiments of the invention are set forth in the description below. Other features, objects and advantages of the invention will be apparent from the following description, including claims.

### DETAILED DESCRIPTION OF THE INVENTION

Reference will now be made to the exemplary embodiments, and specific language will be used herein to describe the same. It should nevertheless be understood that no limitation of the scope of the invention is thereby intended. Alterations and further modifications of the inventive features illustrated herein, which would occur to one skilled in the relevant art and having possession of this disclosure, are to be considered within the scope of the invention. It must be noted that, as used in this specification and the appended claims, the singular forms “a”, “an”, and “the” include plural referents unless the content clearly dictates otherwise. All references including patents, patent applications, and literature cited in the specification are expressly incorporated herein by reference in their entirety.

The invention discloses a method of treating bacterial infections in a subject by administering a compound of Formula (I) or a stereoisomer or a pharmaceutically acceptable derivative thereof.

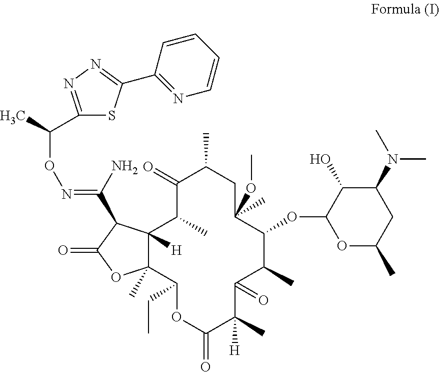 [SMILES: CC[C@@H]1OC(=O)[C@H](C)C(=O)[C@H](C)[C@@H](OC2O[C@H](C)C[C@H](N(C)C)[C@H]2O)[C@](C)(OC)C[C@@H](C)C(=O)[C@H](C)[C@H]2[C@H](/C(N)=N/O[C@@H](C)c3nnc(-c4ccccn4)s3)C(=O)O[C@@]21C]

The term “stereoisomers” as used herein refers to compounds that have identical chemical constitution, but differ with regard to the arrangement of their atoms or groups in space. The compounds of Formula (I) may contain asymmetric or chiral centers and, therefore, exist in different stereoisomeric forms. It is intended, unless specified otherwise, that all stereoisomeric forms of the compounds of Formula (I) as well as mixtures thereof, including racemic mixtures, form part of the present invention. In addition, the present invention embraces all geometric and positional isomers (including cis and trans-forms), as well as mixtures thereof, are embraced within the scope of the invention. In general, a reference to a compound is intended to cover its stereoisomers and mixture of various stereoisomers.

The term “pharmaceutically acceptable derivative” as used herein refers to and includes any pharmaceutically acceptable salt, pro-drugs, metabolites, esters, ethers, hydrates, polymorphs, solvates, complexes and adducts of a compound described herein which, upon administration to a subject, is capable of providing (directly or indirectly) the parent compound. For example, the term “a compound of Formula (I) or a pharmaceutically acceptable derivative thereof” includes all derivatives of the compound of Formula (I) (including pharmaceutically acceptable salts, pro-drugs, metabolites, esters, ethers, hydrates, polymorphs, solvates, complexes and adducts) which, upon administration to a subject, are capable of providing (directly or indirectly) the compound of Formula (I).

The term “pharmaceutically acceptable salt” as used herein refers to one or more salts of a given compound which possesses the desired pharmacological activity of the free compound and which are neither biologically nor otherwise undesirable. In general, the term “pharmaceutically acceptable salts” refer to salts that are suitable for use in contact with the tissues of human and animals without undue toxicity, irritation, allergic response and the like, and are commensurate with a reasonable benefit/risk ratio. Pharmaceutically acceptable salts are well known in the art. For example, S. M. Berge, et al. (J. Pharmaceutical Sciences, 66; 1-19, 1977), incorporated herein by reference in its entirety, describes various pharmaceutically acceptable salts in details. Compound of Formula (I) can be used as such or in the form of its suitable salt. A reference to compound of Formula (I) is intended to include reference to such salts as well.

The term “infection” or “bacterial infection” as used herein includes presence of bacteria, in or on a subject, which, if its growth were inhibited, would result in a benefit to the subject. As such, the term “infection” in addition to referring to the presence of bacteria also refers to presence of normal floras, which are not desirable. The term “infection” includes infection caused by bacteria.

The term “subject” as used herein refers to vertebrate or invertebrate, including a mammal. The term “subject” includes human, animal, a bird, a fish, or an amphibian. Typical, non-limiting examples of a “subject” includes humans, cats, dogs, horses, sheep, bovine cows, pigs, lambs, rats, mice and guinea pigs.

The term “treat”, “treating” or “treatment” as used herein refers to administering a medicament, including a pharmaceutical composition, or one or more pharmaceutically active ingredients, for prophylactic and/or therapeutic purposes. The term “prophylactic treatment” refers to treating a subject who is not yet infected, but who is susceptible to, or otherwise is at a risk of infection (preventing the bacterial infection). The term “therapeutic treatment” refers to administering treatment to a subject already suffering from infection. The terms “treat”, “treating” or “treatment” as used herein also refer to administering compositions or one or more of pharmaceutically active ingredients discussed herein, with or without additional pharmaceutically active or inert ingredients, in order to: (i) reduce or eliminate either a bacterial infection or one or more symptoms of the bacterial infection, or (ii) retard the progression of a bacterial infection or one or more symptoms of the bacterial infection, or (iii) reduce the severity of a bacterial infection or of one or more symptoms of the bacterial infection, or (iv) suppress the clinical manifestation of a bacterial infection, or (v) suppress the manifestation of adverse symptoms of the bacterial infection.

The terms “pharmaceutically effective amount” or “therapeutically effective amount” or “effective amount” as used herein refer to an amount, which has a therapeutic effect or is the amount required to produce a therapeutic effect in a subject. For example, a “therapeutically effective amount” or “pharmaceutically effective amount” or “effective amount” of an antibacterial agent or a pharmaceutical composition is the amount of the antibacterial agent or the pharmaceutical composition required to produce a desired therapeutic effect as may be judged by clinical trial results, model animal infection studies, and/or in vitro studies (e.g. in agar or broth media). Such effective amount depends on several factors, including but not limited to, the microorganism (e.g. bacteria) involved, characteristics of the subject (for example height, weight, sex, age and medical history), severity of infection and particular type of the antibacterial agent used. For prophylactic treatments, a prophylactically effective amount is that amount which would be effective in preventing the bacterial infection.

The term “administration” or “administering” includes delivery of a composition or one or more pharmaceutically active ingredients to a subject, including for example, by any appropriate methods, which serves to deliver the composition or its active ingredients or other pharmaceutically active ingredients to the site of the infection. The method of administration may vary depending on various factors, such as for example, the components of the pharmaceutical composition or the type/nature of the pharmaceutically active or inert ingredients, the site of the potential or actual infection, the microorganism involved, severity of the infection, age and physical condition of the subject and a like. Some non-limiting examples of ways to administer a composition or a pharmaceutically active ingredient to a subject according to this invention include oral, intravenous, topical, intramuscular and parenteral. The compositions according to the invention may also be reconstituted and/or diluted prior to administration.

The compositions according to the invention may further comprise one or more pharmaceutically acceptable excipients. The term “pharmaceutically inert ingredient” or “carrier” or “excipient” refers to a compound or material used to facilitate administration of a compound, for example, to increase the solubility of the compound. Typical, non-limiting examples of such carriers or excipients include bulking agents, solubilizing agents, stabilizing agents, buffering agents, pH adjusting agents, tonicity adjustors, hydrotropic agent, chelating agents, antioxidants, preservatives and the like. Typical, non-limiting examples of solid excipients include, starch, lactose, dicalcium phosphate, sucrose, and kaolin. Typical, non-limiting examples of liquid excipients include sterile water and edible oils such as peanut oil and sesame oil. In addition, various adjuvants commonly used in the art may also be included. These and other such excipients are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press., which is incorporated herein by reference in its entirety.

In one general aspect, there is provided a method of treating a bacterial infection in a subject, said method comprising administering to said subject a compound of Formula (I) or a stereoisomer or a pharmaceutically acceptable derivative thereof in an amount between about 200 mg to about 2000 mg per day, for about 1 to about 10 days.

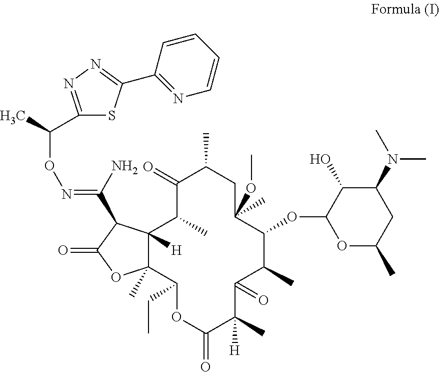 [SMILES: CC[C@@H]1OC(=O)[C@H](C)C(=O)[C@H](C)[C@@H](OC2O[C@H](C)C[C@H](N(C)C)[C@H]2O)[C@](C)(OC)C[C@@H](C)C(=O)[C@H](C)[C@H]2[C@H](/C(N)=N/O[C@@H](C)c3nnc(-c4ccccn4)s3)C(=O)O[C@@]21C]

In some embodiments, there is provided a method of treating a bacterial infection in a subject, said method comprising administering to said subject a compound of Formula (I) or a stereoisomer or a pharmaceutically acceptable derivative thereof in an amount between about 200 mg to about 2000 mg per day, for about 1 to about 10 consecutive days.

In some embodiments, there is provided a method of treating a bacterial infection in a subject, said method comprising administering to said subject a compound of Formula (I) or a stereoisomer or a pharmaceutically acceptable derivative thereof in an amount between about 200 mg to about 2000 mg per day, for about 3 to about 7 days.

In some embodiments, there is provided a method of treating a bacterial infection in a subject, said method comprising administering to said subject a compound of Formula (I) or a stereoisomer or a pharmaceutically acceptable derivative thereof in an amount between about 200 mg to about 2000 mg per day, for about 3 to about 7 consecutive days.

In some embodiments, there is provided a method of treating a bacterial infection in a subject, said method comprising administering to said subject a compound of Formula (I) or a stereoisomer or a pharmaceutically acceptable derivative thereof in an amount between about 400 mg to about 1200 mg per day.

In some embodiments, there is provided a method of treating a bacterial infection in a subject, said method comprising administering to said subject a compound of Formula (I) or a stereoisomer or a pharmaceutically acceptable derivative thereof in an amount between about 400 mg to about 1200 mg per day, for about 1 to about 7 days.

In some other embodiments, there is provided a method for treating a bacterial infection in a subject, said method comprising administering to said subject a compound of Formula (I) or a stereoisomer or a pharmaceutically acceptable derivative thereof in an amount of about 400 mg, 600 mg, 800 mg, 1000 mg, or 1200 mg per day.

In some embodiments, there is provided a method of treating a bacterial infection in a subject, said method comprising administering to said subject a compound of Formula (I) or a stereoisomer or a pharmaceutically acceptable derivative thereof in an amount of about 400 mg per day, for about 3 to about 7 days.

In some embodiments, there is provided a method of treating a bacterial infection in a subject, said method comprising administering to said subject a compound of Formula (I) or a stereoisomer or a pharmaceutically acceptable derivative thereof in an amount of about 600 mg per day, for about 3 to about 7 days.

In some embodiments, there is provided a method of treating a bacterial infection in a subject, said method comprising administering to said subject a compound of Formula (I) or a stereoisomer or a pharmaceutically acceptable derivative thereof in an amount of about 800 mg per day, for about 3 to about 7 days.

In some embodiments, there is provided a method of treating a bacterial infection in a subject, said method comprising administering to said subject a compound of Formula (I) or a stereoisomer or a pharmaceutically acceptable derivative thereof in an amount of about 1000 mg per day, for about 1 to about 7 days.

In some embodiments, there is provided a method of treating a bacterial infection in a subject, said method comprising administering to said subject a compound of Formula (I) or a stereoisomer or a pharmaceutically acceptable derivative thereof in an amount of about 1200 mg per day, for about 1 to about 3 days.

In some embodiments there is provided a method of treating a bacterial infection in a subject, said method comprising administering to said subject a compound of Formula (I) or a stereoisomer or a pharmaceutically acceptable derivative thereof in an amount of about 800 mg per day, for about 3 to about 5 days.

In some embodiments there is provided a method of treating a bacterial infection in a subject, said method comprising administering to said subject a compound of Formula (I) or a stereoisomer or a pharmaceutically acceptable derivative thereof in an amount of about 800 mg per day, for about 3 consecutive days.

In some embodiments, a compound of Formula (I) or a stereoisomer or a pharmaceutically acceptable derivative thereof is administered as oral or parenteral dosage formulation. Typical, non-limiting examples of oral dosage formulation include tablet, capsule, suspension, liquid and the like. Typical, non-limiting examples of parenteral dosage formulation include intravascular, intravenous, intraperitoneal, infusion, subcutaneous, intramuscular injections and the like.

The amount of compound of Formula (I) or a stereoisomer or a pharmaceutically acceptable derivative thereof administered in the subject may vary depending on age, weight sex, medical condition of the subject, type of infection, severity of infection, route and frequency of administration, form of compound of Formula (I) in which it is administered.

The compound of Formula (I) or a stereoisomer or a pharmaceutically acceptable derivative thereof may be administered in different ways. In some embodiments, compound of Formula (I) or a stereoisomer or a pharmaceutically acceptable derivative thereof is administered orally. In some other embodiments, compound of Formula (I) or a stereoisomer or a pharmaceutically acceptable derivative thereof is administered parenterally.

In some embodiments, there are provided methods of treating a bacterial infection in a subject, said method comprising administering to said subject the compound of Formula (I) or a stereoisomer or a pharmaceutically acceptable derivative thereof via parenteral route, followed by administering the compound of Formula (I) or a stereoisomer or a pharmaceutically acceptable derivative thereof via oral route. In some embodiments, there are provided methods of treating a bacterial infection in a subject, said method comprising administering to said subject the compound of Formula (I) or a stereoisomer or a pharmaceutically acceptable derivative thereof via parenteral route for about 1 to about 10 days, followed by administering the compound of Formula (I) or a stereoisomer or a pharmaceutically acceptable derivative thereof via oral route for about 1 to about 10 days. In some embodiments, there are provided methods of treating a bacterial infection in a subject, said method comprising administering to said subject the compound of Formula (I) or a stereoisomer or a pharmaceutically acceptable derivative thereof via parenteral route in an amount of about 200 mg to about 2000 mg per day for about 1 to about 10 days, followed by administering the compound of Formula (I) or a stereoisomer or a pharmaceutically acceptable derivative thereof via oral route in an amount of about 200 mg to about 2000 mg per day for about 1 to about 10 days.

In some embodiments, there are provided methods of treating a bacterial infection in a subject, said method comprising administering to said subject a pharmaceutical composition comprising a compound of Formula (I) or stereoisomer or a pharmaceutically acceptable derivative thereof and one or more pharmaceutically acceptable carriers or excipients.

The compositions according to the invention are useful treating a variety of bacterial infections. Typical, non-limiting examples of infections that can be treated using the compositions according to the invention include those resulting in pneumonia, otitis media, sinusitus, bronchitis, tonsillitis, mastoiditis, pharynigitis, rheumatic fever, glomerulonephritis, respiratory tract infections, skin and soft tissue infections, abscesses and osteomyelitis, puerperal fever, urinary tract infections, urethritis, cervicitis, sexually transmitted diseases, toxin diseases, ulcers, systemic febrile syndromes, Lyme disease, conjunctivitis, keratitis, dacrocystitis, disseminated Mycobacterium avium complex (MAC) disease, gastroenteritis, intestinal protozoa related to infections, odontogenic infections, cough related to infection, gas gangrene related to infection, and atherosclerosis related to infection.

Typical, non-limiting examples of infections in animals that can be treated using compositions according to the invention include bovine respiratory diseases related to infection by Mannheimia haemolytica, Pasteurella multocida, Histophilus somni, Mycoplasma bovis or Bordetella spp.; cow enteric disease related to infection by Escherichia coli or protozoa (i.e., coccidia, cryptosporidia, etc.); dairy cow mastitis related to infection by Staphylococcus aureus, Streptococcus uberis, Streptococcus agalactiae, Streptococcus dysgalactiae, Klebsiella spp., Corynebacterium, or Enterococcus spp.; swine respiratory disease related to infection by Actinobacillus pleuropneumonia, Pasteurella multocida, or Mycoplasma spp.; swine enteric disease related to infection by E. coli, Lawsonia intracellularis, Salmonella, or Serpulinahyodyisinteriae; cow footrot related to infection by Fusobacterium spp.; cow metritis related to infection by Escherichia coli; cow hairy warts related to infection by Fusobacterium necrophorum or Bacteroides nodosus; cow pink-eye related to infection by Moraxella bovis; cow premature abortion related to infection by protozoa (i.e. neosporium); urinary tract infection in dogs and cats related to infection by Escherichia coli; skin and soft tissue infections in dogs and cats related to infection by Staphylococcus epidermidis, Staphylococcus intermedius, Coagulase negative Staphylococci or Pasteurella multocida; and dental or mouth infections in dogs and cats related to infection by Alcaligenes spp., Bacteroides spp., Clostridium spp., Enterobacter spp., Eubacterium, Peptostreptococcus, Porphyromonas, or Prevotella.

In general, the compositions according to the invention are useful in treating infections caused by various microorganisms. In some embodiments, compositions according to the invention are useful in treating infections caused by Staphylococcus spp., Streptococcus spp., Haemophilus spp., Moracella spp., Legionella spp., Chlamydia spp., Clostridium spp. or Mycoplasma spp. Typical, non-limiting examples of Staphylococcus spp. include Staphylococcus aureus, Staphylococcus epedermidis, Staphylococcus saprophyticus and the like. Typical, non-limiting examples of Streptococcus spp. include Streptococcus agalactiae, Streptococcus anginosus, Streptococcus bovis, Streptococcus canis, Streptococcus constellatus, Streptococcus dysgalactiae, Streptococcus equinus, Streptococcus iniae, Streptococcus intermedius, Streptococcus milleri, Streptococcus mitis, Streptococcus mutans, Streptococcus oralis, Streptococcus parasanguinis, Streptococcus peroris, Streptococcus pneumoniae, Streptococcus pseudopneumoniae, Streptococcus pyogenes, Streptococcus ratti, Streptococcus salivarius, Streptococcus tigurinus, Streptococcus thermophilus, Streptococcus sanguinis, Streptococcus sobrinus, Streptococcus suis, Streptococcus uberis, Streptococcus vestibularis, Streptococcus zooepidemicus, Groups C and G streptococci, Viridans streptococci, Groups A, B, and C streptococci, Streptococcal groups C-F (minute-colony streptococci), and the like. Typical, non-limiting examples of Haemophilus spp. include Haemophilus aegyptius, Haemophilus aphrophilus, Haemophilus avium, Haemophilus ducreyi, Haemophilus felis, Haemophilus haemolyticus, Haemophilus influenzae, Haemophilus parainfluenzae, Haemophilus paracuniculus, Haemophilus parahaemolyticus, Haemophilus pittmaniae, Haemophilus segnis, Haemophilus somnus and the like. Typical, non-limiting examples of Moracella spp. include Moracella atlantae, Moracella boevrei, Moracella bovis, Moracella bovoculi, Moracella canis, Moracella caprae, Moracella catarrhalis, Moracella caviae, Moracella cuniculi, Moracella equi, Moracella lacunata, Moracella lincolnii, Moracella nonliquefaciens, Moracella oblonga, Moracella osloensis, Moracella pluranimalium, Moracella porci and the like. Typical, non-limiting example of Legionella spp. include Legionella adelaidensis, Legionella anisa, Legionella beliardensis, Legionella birminghamensis, Legionella bozemanae, Legionella brunensis, Legionella busanensis, Legionella cardiaca, Legionella cherrii, Legionella cincinnatiensis, Legionella donaldsonii, Legionella drancourtii, Legionella dresdenensis, Legionella drozanskii, Legionella dumoffii, Legionella erythra, Legionella fairfieldensis, Legionella fallonii, Legionella feeleii, Legionella geestiana, Legionella gormanii, Legionella gratiana, Legionella gresilensis, Legionella hackeliae, Legionella impletisoli, Legionella israelensis, Legionella jamestowniensis, Legionella jeonii, Legionella jordanis, Legionella lansingensis, Legionella londiniensis, Legionella longbeachae, Legionella lytica, Legionella maceachernii, Legionella massiliensis, Legionella micdadei, Legionella monrovica, Legionella moravica, Legionella nagasakiensis, Legionella nautarum, Legionella oakridgensis, Legionella parisiensis, Legionella pittsburghensis, Legionella pneumophila, Legionella quateirensis, Legionella quinlivanii, Legionella rowbothamii, Legionella rubrilucens, Legionella sainthelensi, Legionella santicrucis, Legionella shakespearei, Legionella spiritensis, Legionella steelei, Legionella steigerwaltii, Legionella taurinensis, Legionella tucsonensis, Legionella tunisiensis, Legionella wadsworthii, Legionella waltersii, Legionella worsleiensis, Legionella yabuuchiae and the like. Typical non-limiting examples of Chlamydia spp. include Chlamydia muridarum, Chlamydia philapecorum, Chlamydia suis, Chlamydia trachomatis, Chlamydia pneumoniae and the like. Typical non-limiting examples of Clostridium spp. include Clostridium diptheriae, Clostridium perfringens and the like. Typical non-limiting examples of Mycoplasma spp. include Mycoplasma amphoriforme, Mycoplasma buccale, Mycoplasma faucium, Mycoplasma fermentans, Mycoplasma genitalium, Mycoplasma hominis, Mycoplasma lipophilum, Mycoplasma orale, Mycoplasma penetrans, Mycoplasma pirum, Mycoplasma pneumoniae, Mycoplasma primatum, Mycoplasma salivarium, Mycoplasma spermatophilum and the like.

In some embodiments, compositions according to the invention are useful in treating infections caused by Peptostreptococcus spp., Actinobacillus haemolyticum, Mycoplasma pneumoniae, Corynebacterium minutissimum, Bartonella henselae; Enterococcus spp., Treponema pallidum, Ureaplasma urealyticum, Neiserria gonorrheae; Helicobacter pylori; Borrelia recurrentis; Borrelia burgdorferi; Listeria spp., Mycobacterium avium complex (MAC) Mycobacterium avium, Mycobacterium intracellulare, Campylobacter jejuni; Cryptosporidium spp.; Bordetella pertussis; Bacteroides spp. and the like.

In some embodiments, there is provided a method of treating community-acquired lower respiratory tract infections. In some embodiments, there is provided a method of treating infections caused by typical extracellular and atypical intracellular bacteria. Typical, non-limiting examples of typical extracellular bacteria are Streptococcus pneumoniae, Haemophilus influenza, Moraxella catarrhalis and the like. Typical, non-limiting examples of atypical intracellular bacteria are Mycoplasma pneumoniae, Chlamydophila pneumoniae, Legionella pneumophila and the like.

It will be readily apparent to one skilled in the art that varying substitutions and modifications may be made to the invention disclosed herein without departing from the scope and spirit of the invention. For example, those skilled in the art will recognize that the invention may be practiced using a variety of different compounds within the described generic descriptions.

### Examples

The following examples illustrate the embodiments of the invention that are presently best known. However, it is to be understood that the following are only exemplary or illustrative of the application of the principles of the present invention. Numerous modifications and alternative compositions, methods and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

Study 1: Study Design for Phase 1 Single Ascending Dose:

A Phase 1 randomised, double-blind, single centre prospective, placebo controlled, sequential cohort study in healthy male and female subjects was conducted. The doses of compound of Formula (I) included for the study were 200 (n=8), 400 (n=12), 600 (n=8), 800 (n=11) and 1200 (n=8) mg to be administered orally as single dose. The blood samples were collected at predose and at various time points up to 24 hours and even at subsequent extended time points to estimate the plasma concentrations of drug. The mean plasma concentration time profile following single ascending oral doses of 200, 400, 600, 800 and 1200 mg to healthy human subjects is illustrated in the FIG. 1.

Study 2: Study Design for Phase 1 Multiple Ascending Dose:

A Phase 1, randomised, double-blind, single center, placebo controlled sequential cohort study in a maximum of 3 cohort of 10 healthy male and/or female subject each was conducted. Subjects received ascending multiple doses of compound of Formula (I) (600, 800 and 1000 mg) or matching placebo once daily on day 1 to day 7. Blood sampling was performed on day 1 and day 7 to determine the drug concentrations in plasma and polymorphonuclear leukocytes (PMN) (FIG. 2 and FIG. 3). FIG. 2 shows the drug concentrations in plasma and polymorphonuclear leukocytes after first day dosing. FIG. 3 shows the drug concentrations in plasma and polymorphonuclear leukocytes after seventh day dosing. Blood sampling was also undertaken at intermittent days to determine the number of doses required to attain the steady state. As depicted in FIGS. 2 and 3, the compound of Formula (I) accumulated more in polymorphonuclear leukocytes (PMN) in comparison to plasma.

Study 3.

Healthy male and female subjects 18 years of age or older who met the study entry criteria were enrolled into the pharmacokinetic study. Each subject received 800 mg (2 tablets of 400 mg) of compound of Formula (I) administered once daily for three days, with 240 ml of drinking water at a consistent time within two hours after a standard meal and direct observation at the study site. Blood samples were collected to measure concentration of compound of Formula (I) in plasma at 3, 6, 9, 12, 24, and 48 hours following the third dose of compound of Formula (I). Similarly, bronchoscopy was done to measure concentration of compound of Formula (I) in epithelial lining fluid (ELF) and alveolar macrophages (AM) 3, 6, 9, 12, 24, and 48 hours following the third dose of compound of Formula (I). The mean (±SD) concentrations of compound of Formula (I) in plasma (total), ELF, and AM at the bronchopulmonary sampling times are illustrated in FIG. 4. Oral administration of compound of Formula (I) at 800 mg produced concentrations that were significantly higher in ELF and AM than simultaneous plasma concentrations throughout the 48-hour period after 3 days of once-daily dosing.

Table 1 gives the mean (±SD) ratios of compound of Formula (I) in ELF or AM to the simultaneous total plasma concentrations. The mean ratios of ELF and AM to simultaneous total plasma concentration for compound of Formula (I) during the 48-hour period after drug administration ranged from 10 to 30 and 270 to 800, respectively. The in vitro activity against common typical and atypical pathogens and the sustained concentration in ELF and AM after the third dose suggest that compound of Formula (I) has the potential to be a useful antibacterial agents for the treatment of lower respiratory tract bacterial infections caused by susceptible pathogens.

TABLE 1
Ratios of ELF or AM to Total Plasma
Concentrations of compound of Formula (I)

| | Sampling | ELF to | AM to |
|---|---|---|---|
| Sr. | Time (hr) | Plasma (Total) | Plasma (Total) |
| 1. | 3 | 17.0 ± 12.0 | 274 ± 187 |
| 2. | 6 | 14.5 ± 8.8 | 378 ± 172 |
| 3. | 9 | 10.4 ± 6.5 | 383 ± 260 |
| 4. | 12 | 12.4 ± 9.3 | 791 ± 614 |
| 5. | 24 | 17.6 ± 6.2 | 486 ± 193 |
| 6. | 48 | 27.2 ± 6.5 | 428 ± 189 |

1. A method of treating a bacterial infection in a subject, said method comprising administering to said subject a compound of Formula (I) or a stereoisomer or a pharmaceutically acceptable derivative thereof in an amount between about 200 mg to about 2000 mg per day, for about 1 to about 10 days.

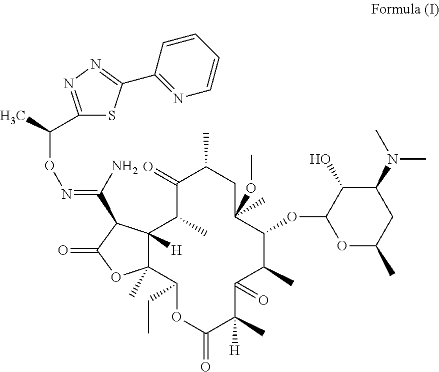 [SMILES: CC[C@@H]1OC(=O)[C@H](C)C(=O)[C@H](C)[C@@H](OC2O[C@H](C)C[C@H](N(C)C)[C@H]2O)[C@](C)(OC)C[C@@H](C)C(=O)[C@H](C)[C@H]2[C@H](/C(N)=N/O[C@@H](C)c3nnc(-c4ccccn4)s3)C(=O)O[C@@]21C]

2. A method according to claim 1, wherein a compound of Formula (I) or a stereoisomer or a pharmaceutically acceptable derivative thereof is administered for about 3 to about 7 days.

3. A method according to claim 1, wherein a compound of Formula (I) or a stereoisomer or a pharmaceutically acceptable derivative thereof is administered for about 3 to about 7 consecutive days.

4. A method according to claim 1, wherein a compound of Formula (I) or a stereoisomer or a pharmaceutically acceptable derivative thereof is administered in an amount between about 400 mg to about 1200 mg per day.

5. A method according to claim 1, wherein a compound of Formula (I) or a stereoisomer or a pharmaceutically acceptable derivative thereof is administered in an amount of about 400 mg, 600 mg, 800 mg, 1000 mg or 1200 mg per day.

6. A method according to claim 1, wherein a compound of Formula (I) or a stereoisomer or a pharmaceutically acceptable derivative thereof is administered in an amount of about 400 mg per day, for about 3 to about 7 days.

7. A method according to claim 1, wherein a compound of Formula (I) or a stereoisomer or a pharmaceutically acceptable derivative thereof is administered in an amount of about 600 mg per day, for about 3 to about 7 days.

8. A method according to claim 1, wherein a compound of Formula (I) or a stereoisomer or a pharmaceutically acceptable derivative thereof is administered in an amount of about 800 mg per day, for about 3 to about 7 days.

9. A method according to claim 1, wherein a compound of Formula (I) or a stereoisomer or a pharmaceutically acceptable derivative thereof is administered in an amount of about 1200 mg per day, for about 1 to about 3 days.

10. A method of claim 1, wherein a compound of Formula (I) or a stereoisomer or a pharmaceutically acceptable derivative thereof is administered in an oral or parenteral dosage form.

11. A method according to claim 1, wherein a compound of Formula (I) or a stereoisomer or a pharmaceutically acceptable derivative thereof is administered in combination with one or more excipients.

12. A method according to claim 2, wherein a compound of Formula (I) or a stereoisomer or a pharmaceutically acceptable derivative thereof is administered in combination with one or more excipients.

13. A method according to claim 3, wherein a compound of Formula (I) or a stereoisomer or a pharmaceutically acceptable derivative thereof is administered in combination with one or more excipients.

14. A method according to claim 4, wherein a compound of Formula (I) or a stereoisomer or a pharmaceutically acceptable derivative thereof is administered in combination with one or more excipients.

15. A method according to claim 5, wherein a compound of Formula (I) or a stereoisomer or a pharmaceutically acceptable derivative thereof is administered in combination with one or more excipients.

16. A method according to claim 6, wherein a compound of Formula (I) or a stereoisomer or a pharmaceutically acceptable derivative thereof is administered in combination with one or more excipients.

17. A method according to claim 7, wherein a compound of Formula (I) or a stereoisomer or a pharmaceutically acceptable derivative thereof is administered in combination with one or more excipients.

18. A method according to claim 8, wherein a compound of Formula (I) or a stereoisomer or a pharmaceutically acceptable derivative thereof is administered in combination with one or more excipients.

19. A method according to claim 9, wherein a compound of Formula (I) or a stereoisomer or a pharmaceutically acceptable derivative thereof is administered in combination with one or more excipients.

20. A method according to claim 10, wherein a compound of Formula (I) or a stereoisomer or a pharmaceutically acceptable derivative thereof is administered in combination with one or more excipients.
